(19) United States
(12) Patent Application Publication
(10) Pub. No.: US 2026/0232354 A1
(43) Pub. Date: Aug. 13, 2026

(54) INSTRUMENT FOR SEMEN INJECTION

(71) Applicant: ONLY STYLE INC., Tokyo (JP)

(72) Inventor: Shintaro ANDO, Tokyo (JP)

(21) Appl. No.: 19/155,425

(22) PCT Filed: Jan. 26, 2024
(86) PCT No.: PCT/JP2024/002364

§ 371 Date: Aug. 11, 2025

(30) Foreign Application Priority Data
Feb. 17, 2023 (JP) 2023-023363
Sep. 1, 2023 (JP) 2023-142297

(51) Int. Cl. A61B 17/435; A61B 17/43

(52) U.S. Cl. CPC A61B 17/435; A61B 17/43

(57) ABSTRACT

A tubular instrument is provided for insertion into a vagina to inject semen (into a body). The tubular instrument has tubular body having tapered leading end portion, and flange-like overhanging portion provided on the outer peripheral surface of leading end portion of tubular body. Overhanging portion is a means for preventing injected semen from leaking out from the vagina. Accordingly, the likelihood of pregnancy using the syringe method is improved without placing an undue burden on a user.

### CROSS REFERENCE TO RELATED APPLICATIONS

This application is a 371 U.S. National Phase of International Application No. PCT/JP2024/002364, filed on Jan. 26, 2024, which claims priority to Japanese Patent Application No. 2023-023363, filed on Feb. 17, 2023 and Japanese Patent Application No. 2023-142297, filed on Sep. 1, 2023. The entire disclosures of the above applications are incorporated herein by reference.

### BACKGROUND

### Technical Field

The present invention relates to a tubular instrument that is advantageous for carrying out a syringe method.

### Related Art

The syringe method is a known method for use by a woman to achieve pregnancy when a man has a problem such as intravaginal ejaculatory dysfunction. The syringe method is a type of fertility treatment, referred to, for example, as a dropper method, an intravaginal injection method or self-AIH, by which semen ejaculated outside a vagina is introduced into a syringe with an attached catheter, and the syringe is inserted into the vagina to inject the semen into the vagina (e.g., see JP 3205250B2 (Patent Literature 1)).

In the conventional syringe method, semen may leak out of the vagina after being injected into the vaginal canal. This is of particular concern for individuals who have a low volume of semen.

It is known that semen remains active in the vagina for about 30 minutes. Retaining as much semen as possible close to the opening of the uterus allows more semen to be delivered into the uterus, thereby increasing a likelihood of pregnancy. To optimize retention of semen, a user may keep her hips in a raised position for an extended period of time. However, this places a heavy burden on the user.

This invention has been made in view of the foregoing circumstances, and provides a means for increasing a likelihood of pregnancy without placing a heavy burden on the user.

### SUMMARY

This invention provides a tubular instrument for insertion into a vagina for injection of semen, and is provided with an overhanging portion for preventing the injected semen from leaking out of the vagina.

In a preferred mode, the present invention provides a tubular instrument for insertion into a vagina of a human for injection of semen, the tubular instrument including: a tubular body having a diameter that is of a sufficient size to prevent entry of the tubular body into a cervical canal of the human, the tubular body having a space formed therein for passage of the semen; a disk shaped overhanging portion provided at a predetermined distance in an axial direction from a leading end of the tubular body, and having a predetermined thickness; and a cylindrical end portion provided between the leading end of the tubular body and the overhanging portion, and having a curved surface protruding in a direction in which the semen is injected.

In another aspect, the present invention provides an instrument for insertion into a vagina of a human for injection of semen, the instrument including: a syringe; and a tubular instrument coupled to a leading end of the syringe, the tubular instrument having: a tubular body that has a diameter of a sufficient size to prevent entry of the tubular body into a cervical canal of the human, the tubular body having a space formed therein for passage of the semen; a disk-shaped overhanging portion provided at a predetermined distance in an axial direction from a leading end of the tubular body and having a predetermined thickness; and a cylindrical end portion provided between the leading end of the tubular body and the overhanging portion and having a curved surface protruding in a direction in which the semen is injected.

In another aspect, the present invention provides a syringe for insertion into a vagina of a human for injection of semen, and having a tubular instrument that serves as an outer cylinder and a plunger, the tubular instrument including: a tubular body having a diameter that is of a sufficient size to prevent entry of the tubular body into a cervical canal of the human, the tubular body having a space formed therein for passage of the semen; a disk-shaped overhanging portion provided at a predetermined distance in an axial direction from a leading end of the tubular body, and having a predetermined thickness; and a cylindrical end portion provided between the leading end of the tubular body and the overhanging portion, and having a curved surface protruding in a direction in which the semen is injected.

### Advantageous Effects of Invention

According to the present invention, the overhanging portion provided on the tubular instrument prevents injected semen from leaking out of the vagina. Accordingly, a likelihood of pregnancy can be increased without placing a heavy burden on the user.

### BRIEF DESCRIPTION OF THE DRAWINGS

FIGS. 1A and 1B are diagrams showing a configuration of a tubular instrument according to a first embodiment of the invention.

FIG. 2 is a diagram showing a first usage form of the tubular instrument.

FIG. 3 is a diagram showing a second usage form of the tubular instrument.

FIG. 4 is a diagram showing an operation according to the embodiment.

FIG. 5 is a diagram showing an operation according to the embodiment.

FIG. 6 is a diagram showing a configuration of a tubular instrument of a second embodiment of the invention.

FIG. 7 is a diagram showing a first form of a tubular instrument of a third embodiment of the invention.

FIG. 8 is a diagram showing a second form of the tubular instrument.

FIG. 9 is a diagram showing a configuration of a tubular instrument according to another embodiment of the invention.

FIG. 10 is a diagram showing a configuration of an overhanging portion of a tubular instrument according to another embodiment (part 1) of the invention.

FIG. 11 is a diagram showing a configuration of a tubular body of the tubular instrument according to the other embodiment.

FIGS. 12A to 12D are a diagram showing a configuration of a tubular body of the tubular instrument according to the other embodiment.

FIG. 13 is a perspective view of a tubular body of a tubular instrument according to another embodiment (part 2) of the invention.

FIG. 14 is a side view of the tubular body of the tubular instrument according to the other embodiment (part 2).

FIG. 15 is a front view of the tubular body of the tubular instrument according to the other embodiment (part 2).

FIG. 16 is a side view in the vicinity of an overhanging portion of a tubular body of a tubular instrument according to yet another embodiment.

FIG. 17 is a cross-sectional view perpendicular to an axial direction of an overhanging portion of a tubular instrument according to yet another embodiment.

FIGS. 18A and 18B include cross-sectional views in the vicinity of the overhanging portion of the tubular instrument shown in FIG. 17, taken along a direction perpendicular to the axis, in which FIG. 18A shows a form before deformation (before insertion into a vagina), and FIG. 18B shows a form after deformation (after insertion into a vagina).

FIG. 19 is a cross-sectional view of syringe 40a.

FIG. 20 is an enlarged cross-sectional view of syringe 40a.

### DETAILED DESCRIPTION

Embodiments of the invention will now be described with reference to the drawings. The term “tubular instrument” as used herein refers to an instrument in which a space is formed that allows passage of semen, at least from one side to another side. The term as used herein is not open to any interpretation that would limit a shape of the instrument.

The term “tubular body” as used herein refers to the main body of the tubular instrument and does not include an overhanging portion, which portion will be described later.

The term “overhanging portion” as used herein means a portion that is provided to reduce leakage of injected semen from a vagina after insertion of the tubular body of the tubular instrument into the vagina and injection of the semen. The term as used herein is not open to any interpretation that would limit a shape or structure of the instrument in absence of description of a specific modifying feature to the contrary. Although the present specification describes several shapes of the overhanging portion as embodiments, those skilled in the art will be able to envisage in view of common technical knowledge in the art various shapes and structures for preventing injected semen from leaking out of a vagina, and all such shapes and structures are encompassed within the scope of the term “overhanging portion” as used herein. Moreover, in place of the term “overhanging portion” another term such as “protrusion” may be used.

Herein, the phrase “for intravaginal insertion” is used to distinguish the tubular instrument of the invention from items such as catheters and devices for artificial insemination that can be inserted not only into a vagina but also into a uterus by way of a cervical canal. That is, the phrase “for intravaginal insertion” as used herein excludes from its scope catheters and other devices that are used for artificial insemination. It is of note that while catheters and other devices for artificial insemination are used as medical equipment by medical personnel during a medical operation or the like, the tubular instrument of the syringe method is for use only at home or a similar place, and thus is significantly different in terms of development, marketing, intended users, [usage] and other aspects.

### First Embodiment

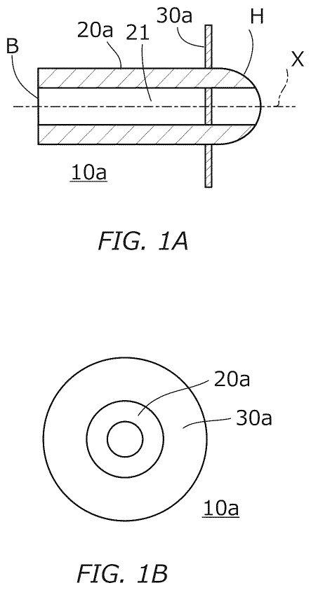

FIG. 1A is a cross-sectional view of tubular instrument 10a of the first embodiment of the invention, cut along a plane including a central axis X. FIG. 1B is a back view of tubular instrument 10a viewed from the base end portion B side.

Tubular instrument 10a is an instrument for insertion into a vagina for injection of semen, and includes a tubular body 20a and an overhanging portion 30a. Preferably, tubular body 20a and overhanging portion 30a are made of a material that can be inserted into the vagina with ease and that minimizes any discomfort or pain for the user. Examples of such a material include resins, such as silicone, elastomers, and polypropylenes. Tubular body 20a and overhanging portion 30a may be integrally molded, or may be separately molded and then adhered to each other. When tubular body 20a and overhanging portion 30a are separately molded, different materials may be used for them. For example, use of a relatively soft material (e.g., a material having a higher elastic modulus) for use in overhanging portion 30a than that used for tubular body 20a makes it possible to realize both ease of insertion and highly close contact between overhanging portion 30a and an inner wall 101m of vagina 101, while minimizing discomfort for a user during insertion. It is of note that a common molding method such as extrusion molding can be employed as the molding method.

Although tubular instrument 10a may be of any thickness, it is preferable that tubular instrument 10a is of a size such that when the user inserts the instrument into her vagina, the leading end of tubular instrument 10a reaches the vicinity of the cervical canal but is unable to enter any further, as is described below. For example, the diameter of tube body 20a of tubular instrument 10a may range from 3 to 20 mm (millimeters), from 5 to 20 mm, and preferably from 5 mm to 15 mm.

The term “diameter” as used herein can be construed as meaning a diameter of a shape that is a perfect circle, a main diameter of a shape that is an oval or a polygon, or a maximum diameter of any other shape. Furthermore, the diameter of the tubular body may vary (in any manner) along the distance from the leading end. For example, the diameter of the tubular body may range from 5 mm to 15 mm on the leading end side of the tubular body from the overhanging portion, and the diameter of the tubular body may range from 10 mm to 25 mm on the base end side from the overhanging portion. Also, from a viewpoint of preventing entry of the tubular instrument into the cervical canal, the maximum diameter of the tubular body on the leading end side from the overhanging portion may be 3.5 mm or more, 4 mm or more, 5 mm or more, 6 mm or more, 7 mm or more, or 8 mm or more.

Here, “maximum diameter” can be construed as meaning a maximum value of the diameter on the leading end side of the tubular body from the overhanging portion. The diameter of overhanging portion 30a may range from 4 mm to 60 mm, preferably from 10 mm to 40 mm, and more preferably 15 to 25 mm. The overhanging portion may also be of any thickness, and the thickness may range from 1 to 20 mm, from 2 to 15 mm, from 3 to 10 mm, or from 4 to 8 mm.

Also, the thickness of the overhanging portion may vary (in any manner) depending on a distance from the axis of the tubular instrument. It is of note that the overhanging portion can be seamlessly integrated with the tubular body by substantially varying the diameter of the tubular body and projecting its outer periphery.

Also, the diameter of a root part of the tubular body may be 25 mm or more. That is, there is no limitation on the upper limit of the diameter of the area that is not inserted into the vagina.

The surface of Leading end portion H of tubular body 20a has a tapered curved shape. A space 21 is formed in tubular body 20a and extends through tubular body 20a along central axis X. Space 21 is a space for passage of semen. Semen is introduced into space 21 from a base end portion B side, and is injected into the vagina from leading end portion H.

Overhanging portion 30a is a means for preventing injected semen from leaking out of the vagina, and is provided on the outer peripheral surface of leading end portion H of tubular body 20a. In this example, overhanging portion 30a has a circular flange shape.

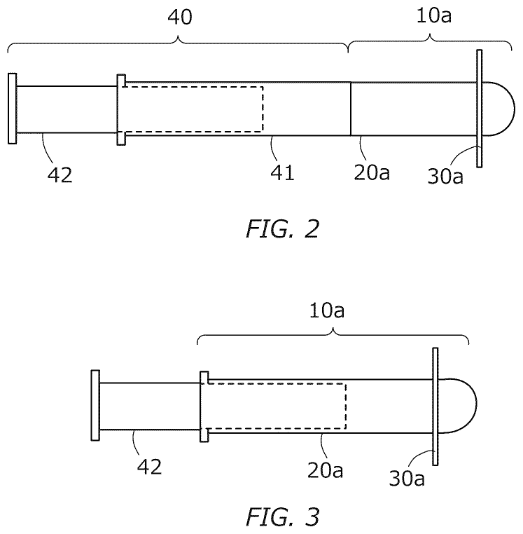

Usage forms of tubular instrument 10a include a first usage form shown in FIG. 2 and a second usage form shown in FIG. 3.

In the first usage form (FIG. 2), base end portion B of tubular instrument 10a is attached to the leading end of syringe 40, which is constituted of an outer cylinder 41 and a plunger 42. The first usage form is a usage form in which tubular instrument 10a is used as a catheter, and is suitable for use with an existing commonly known syringe. Syringe 40 and tubular instrument 10a may be connected in any appropriate manner. For example, in a case where the leading end portion of syringe 40, such as a Luer lock syringe, has a thread on its outer surface, a corresponding thread may be formed on the inner wall surface of tubular body 20a of tubular instrument 10a, and tubular instrument 10a may be fixed by threading it therein from the leading end of syringe 40. Alternatively, in a case that syringe 40 is threadless, tubular instrument 10a may be simply inserted into the leading end portion of syringe 40. In the first usage form, the length of tubular instrument 10a (the length from base end portion B to leading end portion H) may range from 50 to 150 mm, and preferably from 70 to 120 mm.

In the second usage form (FIG. 3), syringe 40 and tubular instrument 10a are integrated. That is, outer cylinder 41 of syringe 40 is replaced with tubular instrument 10a to realize a syringe constituted of plunger 42 and tubular instrument 10a. The second usage form has an advantage in that a catheter is not used, and thus no semen remains in a catheter, thereby preventing wastage of valuable sperm. In the second usage form, tubular instrument 10a also serves as a handle section when inserted into the vagina. Thus, the length of tubular instrument 10a may range from 80 to 250 mm, and preferably from 100 to 200 mm.

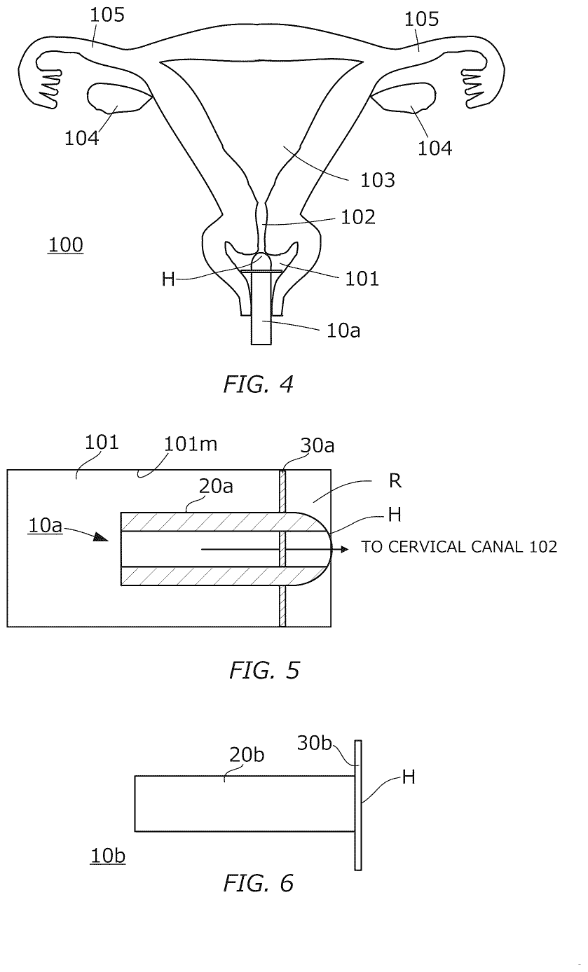

Next, operations of the present embodiment are described with reference to FIGS. 4 and 5. As shown in FIG. 4, female genitalia 100 includes vagina 101, cervical canal 102, uterus 103, ovaries 104, and oviducts 105. Tubular instrument 10a is inserted into vagina 101 with leading end portion H being directed toward cervical canal 102.

FIG. 5 is an enlarged view of tubular instrument 10a inserted into vagina 101. First, tubular instrument 10a is inserted into vagina 101 until leading end portion H reaches the vicinity of the entrance of cervical canal 102. Next, tubular instrument 10a is pulled back slightly (e.g., about 1 cm) in a direction opposite to the insertion direction to form a space R between tubular instrument 10a and the inner wall of vagina 101 on the cervical canal 102 side. Thereafter, plunger 42 (see FIGS. 2 and 3) is depressed to inject semen from leading end portion H toward cervical canal 102.

In the present embodiment, flange-shaped overhanging portion 30a, which expands from the outer periphery of leading end portion H of tubular instrument 10a, reaches inner wall 101m of vagina 101, or comes very close to inner wall 101m. Overhanging portion 30a keeps the injected semen in space R and prevents the semen from leaking out of the inside of vagina 101. Thus, according to the present embodiment, a likelihood of pregnancy can be increased without placing a heavy burden on the user.

### Second Embodiment

FIG. 6 is a side view of tubular instrument 10b of the second embodiment of the invention. Tubular instrument 10b is constituted of tubular body 20b and overhanging portion 30b.

Unlike tubular body 20a of the first embodiment, tubular body 20b has a flat leading end portion H. Except for the shape of this portion, the shape of tubular body 20b is the same as that of tubular body 20a of the first embodiment.

Overhanging portion 30b is a flange-shaped overhanging portion that expands from the outer periphery of leading end portion H of tubular body 20b, similar to overhanging portion 30a of the first embodiment.

As in the first embodiment, tubular instrument 10b may include the first usage form (FIG. 2) and the second usage form (FIG. 3) described above. The same effects as achieved in the first embodiment are also achieved in this embodiment.

### Third Embodiment

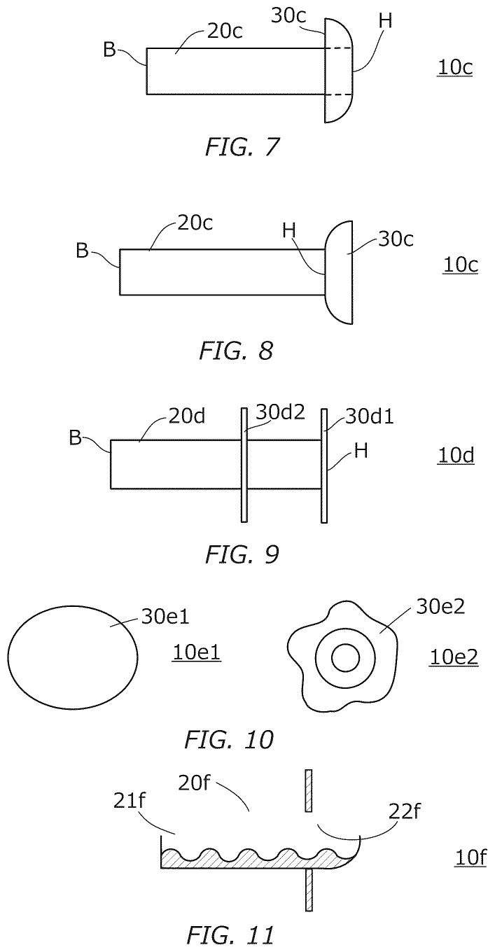

FIG. 7 is a side view showing a first form of tubular instrument 10c of the third embodiment of the invention. FIG. 8 is a side view showing a second form of tubular instrument 10c. Here, the first form is a form of tubular instrument 10c prior to insertion into vagina 101. The second form is a form of tubular instrument 10c when tubular instrument 10c is inserted into vagina 101 until leading end portion H reaches the vicinity of the entrance of cervical canal 102, and thereafter tubular instrument 10c is pulled back slightly (e.g., about 1 cm) in the direction opposite to the above insertion direction (see FIGS. 4 and 5). In this embodiment, overhanging portion 30c is constituted of an elastic member that is deformable into the first form and into the second form.

In the present embodiment, tubular instrument 10c is constituted of tubular body 20c and overhanging portion 30c. In the illustrated example, leading end portion H of tubular body 20c has a flat shape as in the second embodiment. However, leading end portion H of tubular body 20c may alternatively have a tapered shape as in the first embodiment. As in the first and second embodiments, overhanging portion 30c expands from the outer periphery of leading end portion H of tubular body 20c. Also, as in the first and second embodiments, the usage forms of tubular instrument 10c may include the first usage form (FIG. 2) and the second usage form (FIG. 3) described above.

In the first form prior to insertion into vagina 101 (FIG. 7), overhanging portion 30c has a curved surface expanding from leading end portion H toward base end portion B. In the first form, overhanging portion 30c is suitable for insertion of tubular instrument 10c into vagina 101.

When tubular instrument 10c is inserted into vagina 101 until leading end portion H reaches the vicinity of the entrance of cervical canal 102 and thereafter tubular instrument 10c is pulled back slightly in a direction opposite to the insertion direction, overhanging portion 30c changes to the second form (FIG. 8).

In the second form (FIG. 8), overhanging portion 30c assumes a curved surface expanding from leading end portion H toward the side opposite to base end portion B. Overhanging portion 30c keeps the injected semen in space R and prevents the semen from leaking out of vagina 101 (see FIG. 5). Accordingly, leakage of semen out of vagina 101 can be more effectively prevented than in the first embodiment.

### Other Embodiment—Part 1

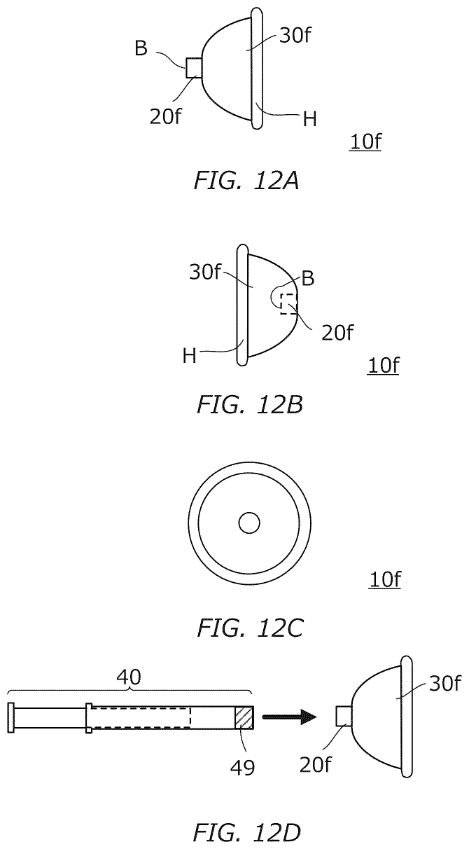

While the first to third embodiments of the present invention have been described above, other embodiments of the invention are conceivable. Examples of such embodiments are described below.
(1) The overhanging portion of the tubular body may be attached at any attachment position, and any number of overhanging portions may be provided. For example, for the attachment position of the overhanging portion, the overhanging portion (in the case of two or more overhanging portions, or any specific overhanging portion) may be provided at a position within 80 mm from the leading end of the tubular body, at a position within 60 mm, at a position within 40 mm, at a position within 20 mm, at a position within 10 mm, at a position within 5 mm, or at a position within 2 mm. Also, the overhanging portion may be provided at a position 2 mm or more away from the leading end of the tubular body, at a position 5 mm or more away, at a position 10 mm or more away, or at a position 20 mm or more away. One, two, or three or more overhanging portions may be attached. Tubular instrument 10d shown in FIG. 9 has overhanging portion 30d1 attached at the foremost end position of leading end portion H of tubular body 20d, and overhanging portion 30d2 attached at a position retracted from the foremost end position toward base end portion B by a predetermined distance.
(2) The shape of the overhanging portion as viewed from the base end portion B side of the tubular body (as viewed from a direction perpendicular to the axis of the tubular body) need not be circular. Tubular instrument 10e1 shown in FIG. 10 has overhanging portion 30e1 in an oval shape. Tubular instrument 10e2 shown in FIG. 10 has overhanging portion 30e2 with an uneven outer periphery. According to these modes, the overhanging portion is able to come into more intimate contact with the vaginal inner wall. In addition, the shape of the overhanging portion as viewed from the base end portion B side of the tubular body may be, for example, a square shape or any other polygonal shape.
(3) As with tubular instrument 10f shown in FIG. 11, a helical groove 22f may be formed in the inner wall surrounding space 21f for passage of semen in tubular body 20f. As a result of flow of semen being regulated by groove 22f, it is possible to reduce turbulence resistance and frictional resistance, and to accelerate flow.
(4) The preferable sizes of the overhanging portion and the tubular body are as described in the first embodiment, while the shape and size of overhanging portion 30 and its relative size and position with respect to tubular body 20 may be set as appropriate. For example, tubular instrument 10f may overall have a bell shape, an umbrella shape, or a cup shape ; and tubular body 20f, which is shorter than overhanging portion 30, may be provided at a central part of the cup shape. For example, tubular instrument 10f shown in FIG. 12 may be employed. FIG. 12A shows a shape upon injection of semen, and FIG. 12B shows a shape upon insertion (the insertion direction being from left to right as shown in the figure). FIG. 12C is a view of FIG. 12A taken from directly above. Overhanging portion 30 is formed from an elastic body. Thus, overhanging portion 30 deforms from the state during insertion to the state during ejaculation of semen, centered on tubular body 20f. The resulting deformation is similar to that of an umbrella turned inside out. Tubular instrument 10f is connected to syringe 40 by fitting thread 49 formed on the peripheral surface (inner wall surface or outer wall surface) of the leading end portion of syringe 40 to a thread formed on the peripheral surface (outer wall surface or inner wall surface) of tubular body 20f, as shown in FIG. 12D. In the present embodiment, the diameter of tubular body 20f may range from 3 to 20 mm (millimeters), and preferably from 4 mm to 10 mm. Also, the diameter of overhanging portion 30f (the diameter of its largest portion) may range from 10 mm to 60 mm, and preferably from 20 mm to 40 mm. In the present embodiment, syringe 40 serves as a handle when inserted into the vagina, and the length of tubular body 20f may range from 1 to 20 mm, and preferably from 2 mm to 10 mm.

### Other Embodiments—Part 2

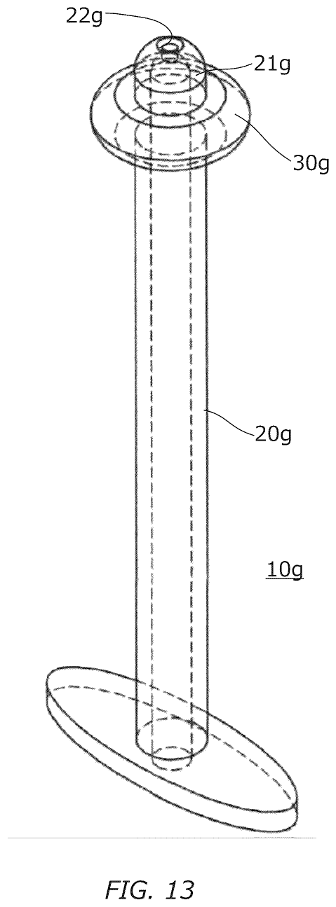

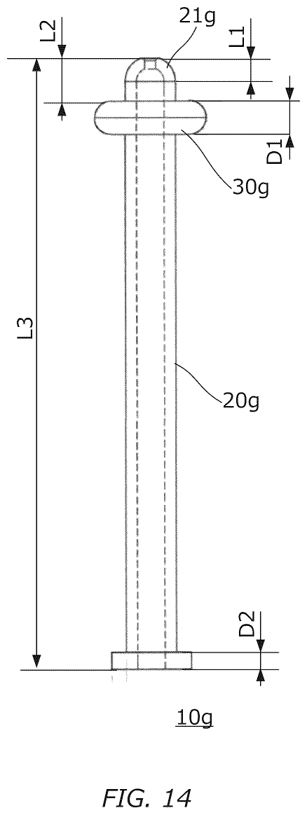

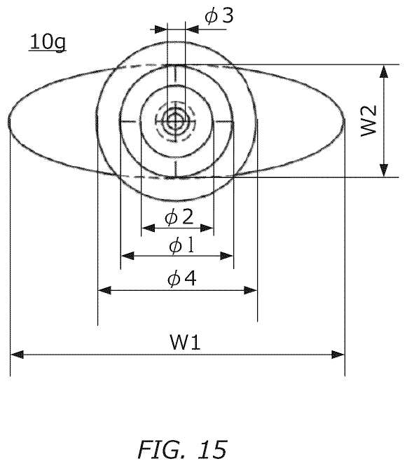

Tubular instruments according to yet other embodiments are now described with reference to FIG. 13 to FIG. 15. FIG. 13 is a perspective view of tubular instrument 10g, FIG. 14 is a front view thereof, and FIG. 15 is a plan view thereof. As shown in these figures, tubular instrument 10g is a syringe having a length L3, with a basic structure constituted of an outer cylinder having an outer diameter φ1 and a plunger with an oval disk shaped rear end portion that has a length W1 in a lengthwise direction and a length W2 in a widthwise direction. Tubular instrument 10g is integrally molded using, for example, an elastic resin material. The function of injecting semen introduced from an end of the tube into the vagina is the same as that of the tubular instruments according to the above-described embodiments. It is of note that FIGS. 13 to 15 show a state in which a depressible member has been depressed to its maximum extent.

More specifically, tubular instrument 10g includes tubular body 20g having a tubular shape whose outer diameter is φ1, and having a space (inner diameter φ2) formed for passage of semen to the inside, and overhanging portion 30g formed to overhang outward from the peripheral surface of tubular body 20g. Cylindrical end portion 21g is formed from the leading end of tubular body 20g to distance L1 in the axial direction (hereinafter simply referred to as a distance). At least a part of cylindrical end portion 21g has a rounded shape (e.g., a substantially semi-spherical shape) with a curved surface protruding toward the leading end portion (with respect to the direction in which semen is injected (semen injection direction)). It is of note that semen injection opening 22g of cylinder end portion 21g has a cone shape with an inner wall expanding outward so that a semen injection direction is regulated.

In this mode, overhanging portion 30g having outer diameter φ4 is formed from distance L2 from the leading end portion to distance L2+D1. Overhanging portion 30g prevents semen injected into the vagina from leaking outside of the vagina, similar to the above-described embodiments. Overhanging portion 30g is axially symmetrical and has a rounded, substantially disk shape (donut shape, floating ring shape). The curvature is set, for example, to 3 mm.

By employing such a shape pain and other discomfort felt by a user is minimized when the tubular instrument is inserted into the vagina. Further, the internal space of cylindrical end portion 21g is tapered (the diameter changes from φ2 to φ3). As a result, a pressure is increased immediately before semen is injected, and a speed and directional accuracy is increased when the semen is injected.

Following are examples of sizes.
L3: 100 to 150 mm (millimeters), more preferably 105 to 115 mm
L2: 6 to 10 mm, more preferably 7.5 to 8 mm
L1: 3 to 6 mm
D1: 4 to 8 mm
φ1: 9 to 15 mm
φ2: 5 to 10 mm
φ3: 1 to 3 mm
φ4: 15 to 25 mm, more preferably 19 to 21 mm
W1: 35 to 50 mm
W2: φ1+2 mm to φ1+6 mm

The above sizes can be adopted as appropriate, based on a shape of a vagina, an amount of semen, or the like. For example, L2 relates to a size of a region in which injected semen remains in the vagina. Thus, if the value of L2 is too large, a likelihood of the injected semen entering the cervical canal decreases; on the other hand, if the value is too small, a likelihood of flooding of the semen increases. Further, if φ4, which relates to semen retention capacity, is too large, discomfort upon insertion of the tubular instrument will increase; one the other hand, if it is too small, the tubular instrument will not come into sufficiently close contact with the vaginal inner wall (gaps will be present) . Furthermore, if L3 is too large, it will become difficult to handle the plunger, whereas if it is too small, the entire instrument may enter the vagina, thereby making it difficult to operate the plunger. It is, of course, possible to make various modifications to the present embodiment by combining or adapting the foregoing embodiments.

In summary, as described above, the present invention is a tubular instrument suitable for insertion into a vagina for injection of semen, and is provided on its outer peripheral surface with an overhanging portion for preventing injected semen from leaking out of the vagina. The overhanging portion is not limited to one having a shape that protrudes in a direction perpendicular to the direction of semen injection (in the direction in which the plunger is depressed), and, alternatively, may have a paraboloid or any other curved surface, and need only have a structure in which the overhanging portion expands in the radial direction of the tubular instrument into which semen is injected in order to reduce semen injected into the vagina passing through a gap with the inner wall of the vagina and moving (leaking) in the opposite direction (the direction toward the outside of the vagina). Note that the direction in which the overhanging portion extends may be perpendicular to the axial direction (i.e., horizontal direction), or may be at any other angle (i.e., obliquely upward direction or obliquely downward direction relative to the axial direction).

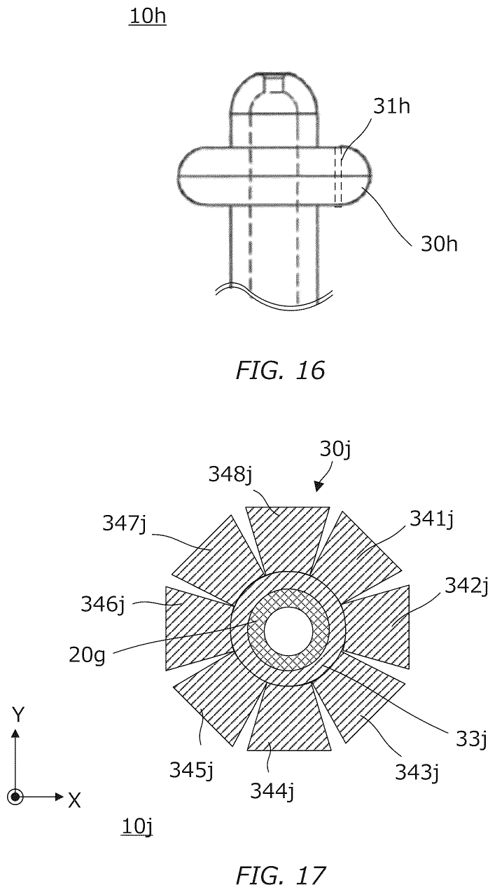

Further, as described above, the overhanging portion is provided in order to reduce leakage of injected semen to the outside of the vagina. Thus, the overhanging portion may have a shape with a cutout of a part thereof, or may have a shape that includes one or more holes (of any size) for air venting or another purpose. For example, tubular instrument 10h shown in FIG. 16 has overhanging portion 30h in which through-hole 31h for air venting is formed in the axial direction. This allows for smooth vaginal insertion and removal of tubular instrument 10h. It is of note that the cutout portion can be formed, for example, as a groove extending in the axial direction from the upper end to the lower end of the overhanging portion, and may have a three-dimensional shape and size.

Further, a structure is also conceivable that includes a plurality of pinwheel-like or radial plate-shaped blades expanding from the center to the outer periphery, and which curve inside the vagina and are integrated to form a cup shape and prevent leakage of semen. Specifically, as shown in FIG. 17, tube body 20j of tubular instrument 10j has overhanging portion 30j. Overhanging portion 30j is constituted of annular member 33j and eight plate-shaped blade portions 341j to 348j formed radially on a surface (XY plane) perpendicular to the axial direction (Z-axis direction) from annular member 33j. Blade portions 341j to blade portion 348j are formed of an elastic material such as resin.

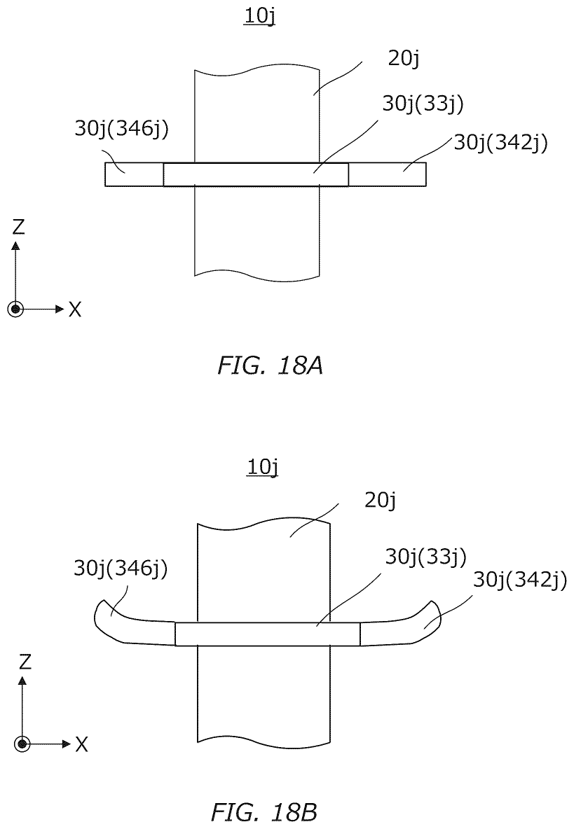

FIG. 18 includes cross-sectional views of tubular instrument 10j in the vicinity of a region where overhanging portion 30j is formed. FIG. 18A shows a state before insertion (a state where no force is applied to overhanging portion 30j). As shown in the figure, flat plate-shaped blade portions 341j to 348j, each of which has thickness D2, extend in a direction perpendicular to the axis (in the XY-plane) in a state where no force is applied. FIG. 18B shows tubular instrument 10j inserted by the user into the vagina in the +Z direction (from bottom to top in the figure) and pulled back slightly from the deepest position. In this state, edge surfaces of blade portions 341j to 348j are in contact with the vaginal wall (not shown), and as a result of blade portions 341j to 348j bending in the +Z direction, the blade portions overlap each other to fill gaps between the blade portions present in the state before deformation, and blade portions 341j to 348j are integrated to form a cup-like, upward protruding curved surface (paraboloid). This curved surface serves as a receiving tray for injected semen, and prevents the semen from leaking out of the vagina (−Z direction).

In the case where the syringe is constituted of the tubular instrument as an outer cylinder and a plunger as a depressible member as shown in FIG. 3, and in the case where a plunger is inserted into another tubular instrument, it is desirable that the shape of the plunger and the shape of the tubular instrument be designed such that as little semen as possible remains inside the syringe. For this purpose, for example, a configuration may also be employed in which the inner shape and the shape of the opening of the tubular instrument, and the shape of the leading end portion of the plunger for injection of semen are formed such that these portions engage with each other without any clearance therebetween.

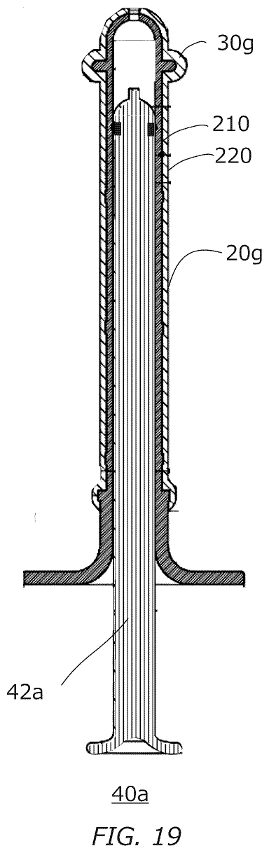

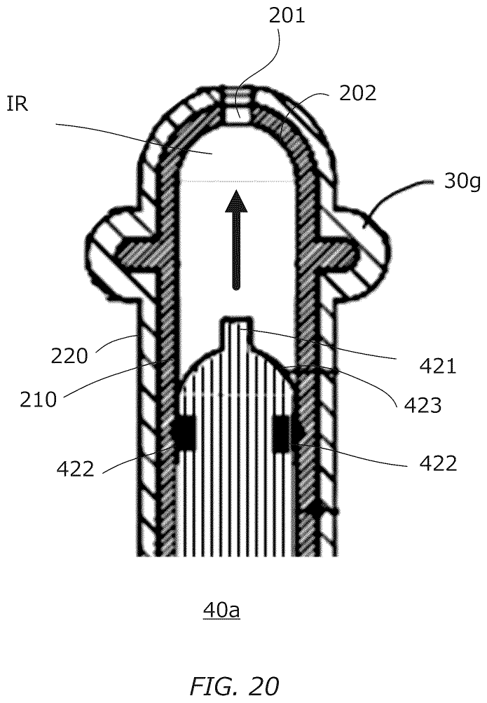

FIG. 19 is a cross-sectional view of syringe 40a constituted of tubular instrument 10g as an outer cylinder and plunger 42a as a depressible member. FIG. 20 is an enlarged view of the vicinity of the leading end of the syringe. Syringe 40a is different from syringe 40 shown in FIG. 3 in that, as shown in FIG. 20, a substantially semi-spherical portion 423 and a substantially cylindrical protrusion 421 at a central portion thereof are formed at a leading end of plunger 42a that is located above O-ring 422. A structure is provided in which, when plunger 42a is depressed to its maximum extent, substantially semi-spherical portion 423 and substantially semi-spherical inner wall 202 of tubular instrument 10g come into highly close contact with each other without any clearance therebetween, and fit into opening 201 provided for injecting semen in tubular instrument 10g (protrusion 421 is inserted into the opening without clearance). This allows the semen present in space IR to be injected without leaving any residue.

The tubular instrument of the present invention, including the tubular body and the overhanging portion, may be constituted of an inner component and an outer component, and these components may be made of separate materials. For example, in the embodiment shown in FIGS. 19 and 20, inner component 210 of tubular instrument 10g is made of a plastic material, e.g., polyolefin such as polyethylene or polypropylene, and outer component 220 covering the outside of inner component 210 is made of a flexible and elastic material friendly to the skin and mucous membrane, e.g., a thermoplastic elastomer such as silicone rubber or urethane rubber. With this configuration, the inner component is not bent by external force, so that it comes into sufficiently intimate contact with the plunger, and the plunger can be smoothly inserted. Further, the outer component is sufficiently flexible and elastic such that it cannot cause any damage to the vagina. The outer component need only be provided at a portion that is substantially inserted into the vagina, and the inner component may be exposed to the outer surface, for example, at a gripping portion of the tubular instrument, as shown in FIG. 19. Further, although the inner component is also provided at the overhanging portion in FIG. 19, the overhanging portion may be formed only of the outer component in order to impart flexibility to the overhanging portion.

It is of note that in the embodiments introduced so far, the base end side of the tubular body is not provided with any other portion or is only provided with a small protrusion as in the first and second usage forms shown in FIGS. 2 and 3. However, variations can be envisaged, such as providing a gripping portion to serve as a handle when operating the tubular instrument of the invention, a cover portion for covering the syringe attached to the tubular instrument, or portions having various other functions. These portions are provided to add functions different from that of the tubular body for insertion into the vagina. Thus, it is possible to disregard these portions when referring to the configuration of the tubular body herein (e.g., when considering the configuration in which “the diameter of the tubular body ranges from 3 to 20 mm” herein, a mode in which the diameter of the gripping portion integrated with the tubular portion is 35 mm can also be construed as satisfying the configuration if the diameter of the tube body excluding the gripping portion is 15 mm, for example). Each of the embodiments described so far can be combined with each other, taking into consideration the descriptions in the present specification and common technical knowledge in the art, and it will be understood that all embodiments in which part of the configuration of a particular embodiment is combined with another embodiment are also described herein.
(5) In all of the embodiments, it is preferable that leading end portion H has a through-hole (not shown) that functions as an air escape path. In this way, a likelihood is reduced that semen will leak together with air when semen is injected into the vagina.

1. A tubular instrument for insertion into a vagina of a human to inject semen, the tubular instrument comprising:
a tubular body having a diameter that is of a sufficient size to prevent entry of the tubular body into a cervical canal, the tubular body having a space formed therein for passage of the semen;
a disk-shaped overhanging portion provided at a predetermined distance in an axial direction from a leading end of the tubular body and having a predetermined thickness; and
a cylindrical end portion provided between the leading end of the tubular body and the overhanging portion and having a curved surface protruding in a direction in which the semen is injected.

2. The tubular instrument according to claim 1,
wherein the tubular body has a diameter of 5 mm or more.

3. The tubular instrument according to claim 1,
wherein the overhanging portion has a diameter ranging from 10 mm to 40 mm.

4. The tubular instrument according to claim 1,
wherein the overhanging portion is formed of an elastic member deformable into a first form in which a curved surface expanding from a leading end portion toward a base end portion of the tubular instrument is formed, and a second form in which a curved surface expanding from the leading end portion toward a side opposite to the base end portion is formed.

5. The tubular instrument according to claim 1,
wherein a helical groove is formed on an inner wall forming the space for passage of the semen.

6. An instrument for insertion into a vagina of a human to inject semen, the instrument comprising:
a syringe; and
a tubular member coupled to a leading end of the syringe,
the tubular member having:
a tubular body having a diameter of a sufficient size to prevent entry of the tubular body into a cervical canal, the tubular body having a space formed therein for passage of the semen;
a disk-shaped overhanging portion provided at a predetermined distance in an axial direction from a leading end of the tubular body and having a predetermined thickness; and
a cylindrical end portion provided between the leading end of the tubular body and the overhanging portion and having a curved surface protruding in a direction in which the semen is injected.

7. A syringe for insertion into a vagina of a human to inject semen, and having a tubular member that serves as an outer cylinder and a plunger,
the tubular member comprising:
a tubular body having a diameter that is of a sufficient size to prevent entry of the tubular body into a cervical canal, the tubular body having a space formed therein for passage of the semen;
a disk-shaped overhanging portion provided at a predetermined distance in an axial direction from a leading end of the tubular body and having a predetermined thickness; and
a cylindrical end portion provided between the leading end of the tubular body and the overhanging portion and having a curved surface protruding in a direction in which the semen is injected.

8. The syringe according to claim 7,
wherein the plunger has a protrusion capable of fitting into an opening for injecting the semen formed at a leading end portion of the tubular member.
